(19) United States
(12) Patent Application Publication
(10) Pub. No.: US 2010/0304428 A1
(43) Pub. Date: Dec. 2, 2010

(54) CELL HOLDING METHOD, CELL TESTING METHOD AND CELL HANDLING APPARATUS

(75) Inventors: Tsuyoshi Nomoto, Tokyo (JP); Kohei Watanabe, Yokohama-shi (JP); Takeshi Miyazaki, Yokohama-shi (JP); Toshio Tanaka, Tsu-shi (JP); Yasuhito Shimada, Nagoya-shi (JP)

Correspondence Address: CANON U.S.A. INC. INTELLECTUAL PROPERTY DIVISION, 15975 ALTON PARKWAY, IRVINE, CA, 92618-3731, US

(73) Assignee: CANON KABUSHIKI KAISHA, Tokyo (JP)

(21) Appl. No.: 12/788,178

(22) Filed: May 26, 2010

(30) Foreign Application Priority Data
May 29, 2009 (JP) 2009-131625

(51) Int. Cl. C12Q 1/02; C12N 5/00; C12M 1/00; C12N 11/00

(52) U.S. Cl. 435/29; 435/325; 435/308.1; 435/174

(57) ABSTRACT

It is intended to provide a method for individually arranging and holding, with rapidity and few variations, the number of cells required for bioassay. The present invention provides a cell holding method including: preparing a sheet provided with a plurality of through-holes; and bringing a suspension liquid of cell-supported particles into contact with the sheet, wherein each of the holes has a size that permits only one of the particles to be held therein together with a liquid.

### BACKGROUND OF THE INVENTION

1. Field of the Invention

The present invention relates to a cell holding method, a cell testing method and a cell handling apparatus, which are useful for arranging cells and efficiently performing procedures such as test and fractionation.

2. Description of the Related Art

In the field of drug development or disease diagnosis, large-scale and rapid bioassay systems have been demanded, which reliably evaluate chemical substances or biologically active substances for their influences on cells with high throughputs.

For large-scale and rapid handling using the bioassay systems, a population having the number of cells suitable for a detection approach must be arranged at determined positions, and predetermined procedures such as administration of predetermined substances, liquid exchange, measurement and fractionation must be conducted efficiently on the cell population.

Heretofore, high-density (384- or 1536-well) micro-well plates have been developed and put on the market for handling a cell population in bioassay and performing such bioassay with high throughputs.

Cells can be classified according to their properties into floating cells, adherent cells and spheroids (cell aggregates). In bioassay targeting adherent cells using a micro-well plate, these adherent cells are temporarily converted into a floating form by, for example, trypsin treatment, and a suspension liquid containing an appropriate number of the resulting cells is then dispensed to each well. Then, the cells are left standing under predetermined environmental conditions and cultured until the number of cells reaches that suitable for bioassay detection conditions.

However, such bioassay of adherent cells using a high-density micro-well plate faces the challenge of dispensing, to each well with few variations, a suspension liquid of the cells converted in a floating form by trypsin treatment. When a 1536-well plate is used, each well has a capacity as small as 2 to 10 μl. On the other hand, the adherent cells converted in a floating form by trypsin treatment need to be grown by culture without becoming confluent. Therefore, a high-density cell suspension liquid cannot be dispensed from the beginning. Specifically, the challenge of the high-density micro-well plate in use is to certainly dispense a suspension liquid of a small number of adherent cells with few variations to each well.

Alternatively, bioassay targeting floating cells (e.g., blood cells) or spheroids may be conducted using a micro-well plate. In such a case, exchange of liquids in wells requires the procedure of temporarily precipitating a cell population by centrifugation and then exchanging the supernatant liquid. This is responsible for a reduction in the throughput of bioassay using a micro-well plate.

The throughput of such bioassay using a micro plate can be improved by known methods such as those using a filter instead of a well plate and those using micro-fluidic channels. National Publication of International Patent Application No. 2008-538509 discloses an example of a cell holding method using filter pores of size smaller than that of cells, wherein the cells are held not in the pores but on the surfaces of the filter pores. National Publication of International Patent Application No. 2004-510996 discloses an example of holding cells in an array with tapered through-holes.

However, the cell holding method using a filter, described in National Publication of International Patent Application No. 2008-538509, has the problem of cell contamination between the pores because the cells on the filter are not placed in the pores. The method also presents the problem that only a small number of cells are held on each through-hole because the cells are almost equal in size to the through-holes.

The method for holding cells in an array with tapered through-holes, described in National Publication of International Patent Application No. 2004-510996, has the problem that the number of cells held in each through-hole is smaller than that required for subsequent assay because the cells are almost equal in size to the through-holes.

Thus, an object of the present invention is to provide a method for individually arranging and holding, with rapidity and few variations, the number of cells required for bioassay.

### SUMMARY OF THE INVENTION

A cell holding method of the present invention includes: preparing a sheet provided with a plurality of through-holes; and bringing a suspension liquid of cell-supported particles into contact with the sheet, wherein each of the holes has a size that permits only one of the particles to be held therein together with a liquid.

A cell handling apparatus of the present invention includes a holding unit for holding a sheet provided with a plurality of through-holes and further includes at least one of the following units (A) to (G): (A) an arranging unit for arranging cell-supported particles to the holes in the sheet; (B) a controlling unit for controlling an environment surrounding the sheet to a predetermined environment; (C) an applying unit for applying a droplet to the holes in the sheet; (D) an observing unit for observing the states of the particles held in the holes in the sheet; (E) a transporting unit for transporting, from the sheet, the particles held in the holes in the sheet; (F) a recognizing unit for recognizing the positions of the holes in the sheet; and (G) a liquid exchange unit for performing liquid exchange in the holes in the sheet.

The present invention also provides a kit for cell test or culture, including: a sheet provided with a plurality of through-holes; and particles for supporting cells, wherein each of the holes has a size that permits only one of the particles to be held therein together with a liquid.

The method of the present invention enables the number of cells required for bioassay to be individually arranged and held with rapidity and few variations and to be efficiently cultured and tested. The apparatus of the present invention enables cells to be individually arranged and to be efficiently analyzed and fractionated.

Further features of the present invention will become apparent from the following description of exemplary embodiments with reference to the attached drawings.

### BRIEF DESCRIPTION OF THE DRAWINGS

FIG. 1A is a diagram illustrating a sheet (particle holding sheet) provided with a plurality of through-holes, and FIG. 1B is a cross-sectional view and a plane view schematically illustrating the state of a particle held in each hole in the sheet provided with a plurality of through-holes.

FIG. 2 is a plane view schematically illustrating the state of droplets of a biologically active substance solution uniformly discharged from an inkjet portion to particles held in the holes.

FIGS. 3A and 3B are partially enlarged views illustrating two aspects of a particle holding sheet provided with a liquid exchange site, and FIG. 3C is a two-dimensional view of a particle holding sheet provided with a liquid exchange site.

FIG. 4 is a plane view of a particle holding sheet used in Examples.

FIG. 5 is an image of a particle holding sheet taken in Example 1.

FIG. 6 is an image of a cell on a particle taken in Example 1.

FIG. 7 is a schematic view of the system configuration of a handling apparatus used in Examples.

### DESCRIPTION OF THE EMBODIMENTS

Preferred embodiments of the present invention will now be described in detail in accordance with the accompanying drawings.

Hereinafter, the embodiments of the present invention will be described with reference to the drawings. The embodiments individually disclosed herein are examples of a cell holding method, a cell testing method and a cell handling apparatus of the present invention, and the present invention is not limited thereto.

### First Embodiment

A cell holding method of the present invention includes: preparing a sheet provided with a plurality of through-holes 2; and bringing a suspension liquid of cell-supported particles 3 into contact with the sheet, wherein each of the holes 2 holds only one of the particles 3.

The cells used in the present invention refer to, for example, adherent cells, floating cells and spheroids (cell aggregates). Specific examples thereof include human cervical cancer cell line cells, nerve cells, liver cells, fibroblasts, myoblasts, smooth muscle cells, myocardial cells, skeletal muscle cells, stem cells, mesodermal stem cells, embryonic stem cells, glial cells, fetal stem cells, hematopoietic stem cells, mast cells, fat cells, neural stem cells and blood cells. In the present invention, the particles 3 can support one cell type on a sheet-by-sheet basis for the purpose of holding, testing and handling cells used mainly in bioassay. This enables the number of cells of single type required for bioassay to be individually arranged and held with rapidity and few variations and to be efficiently cultured and tested.

However, the present invention is not limited thereto, and two or more cell types may be supported in one sheet. For supporting different cell types in one sheet, the holding region of the sheet is sectioned, and supported particles differing in cell type can be arranged in the sections different from each other. Alternatively, the particles may be arranged randomly without sectioning the region. In such a case, the particles are separately labeled (e.g., stained), and the cells supported thereby may be differentiated with the labels. One particle may also support two or more cell types.

The particles 3 used in the present invention are not limited as long as the particles 3 are capable of supporting the cells. Examples of a material for the particles 3 include organic polymer substances and inorganic substances. Examples of the organic polymer substances include acrylic acid polymers, styrene polymers, methacrylic acid polymers, alginate hydrogels, collagen-derived peptide hydrogels and agarose gels. Examples of the inorganic substances include silica, alumina, hydroxyapatite and magnetic iron oxide.

The material for the particles 3 can be a material excellent in light transmission in terms of microscopic observation or can be an autofluorescence-free material in terms of fluorescent microscopic observation.

In the present invention, the use of the cell-supported particles 3 enables the cells to be rapidly arranged. The cell-supported particles 3 can be prepared by methods including: a method which includes culturing adherent cells in the presence of particles 3; a method which includes adding, into a culture solution of floating cells, particles 3 capable of adsorbing the floating cells; and a method which includes adding polymerizable monomer substances into a culture solution of floating cells or spheroids and polymerizing the substances into particles so as to entrap and immobilize the cells or spheroids therein.

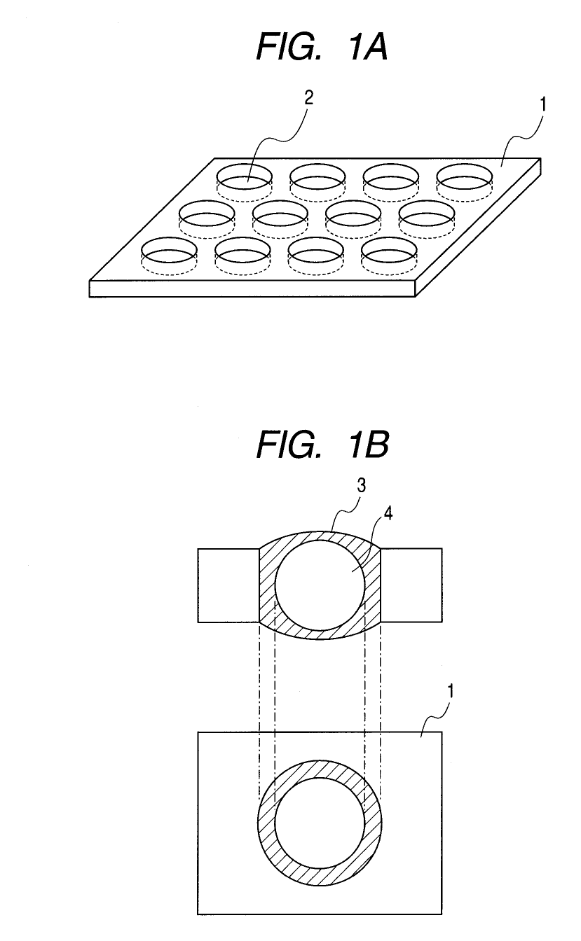

FIGS. 1A and 1B illustrate one example of a sheet (hereinafter, also referred to as “particle holding sheet”) provided with a plurality of through-holes 2 used in the present invention. The sheet has a base (substrate 1) and a plurality of through-holes (mouths) 2 designed to be capable of holding particles 3 together with a liquid 4 (see FIG. 1B). Each hole 2 has a size that enables only one particle 3 to be held and does not permit two particles 3 to be held. Presumably, the liquid 4 is held by its interaction with (adhesion to) the inner wall of each hole in the sheet, and each particle 3 is wrapped in the liquid 4 and held in this state by the surface tension of the liquid.

As an example thereof, FIG. 1B is a cross-sectional view and a plane view schematically illustrating the state of the particle 3 held in the hole 2 together with the liquid 4. In this context, the particle 3 is almost spherical in shape for the hole 2 having round mouths.

Any material in which the holes 2 can be formed can be used for the base 1. Examples of the material that can be used include: metals such as iron, copper and aluminum, or alloys containing these metals; ceramics such as glass, alumina and silicon; plastic resins such as Teflon (registered trademark), polyethylene, polypropylene, polyester, polyacetal, silicon rubbers, polycarbonate, polyvinyl chloride, polystyrene and nylon; and composite materials thereof. However, the base 1 can basically be made of a material that is neither dissolved in water nor elutes its ingredients by water. The holes 2 in the sheet may have a hydrophilized or rough-surfaced inner wall so as to easily hold the liquid 4. A material for the surface of the sheet is not particularly limited in its color and can be a material that suppresses light reflection in terms of microscopic observation or can be an autofluorescence-free material in terms of fluorescent microscopic observation.

In the present invention, each hole 2 must have an appropriate aperture area and an appropriate thickness, compared with the size of the particle 3. Specifically, the size (aperture area and thickness) of the hole 2 largely influences the efficient and stable holding of only one particle 3 in each hole 2 together with the liquid 4.

The holes 2 may have any shape such as polygonal (e.g., quadrangular), elliptical and star shapes, in addition to a circular shape illustrated in FIGS. 1A and 1B.

The aperture area of each hole 2 can fall within a range of 1.05 times to 2.63 times the maximum cross-sectional area of each particle 3 to be held therein. For stably holding one particle 3 in each hole 2 with higher probability, each hole 2 can particularly have an aperture area that falls within a range of 1.2 to 2.25 times the maximum cross-sectional area of the particle 3. The maximum cross-sectional area of the particle 3 refers to the area of a section passing through the center of gravity of, for example, a sphere or almost sphere. The sheet can usually be kept horizontally in holding the particles 3.

The size that does not permit two or more particles to be held refers to a size that does not permit the maximum sections of two particles to be geometrically arranged at least relative to the hole 2. In a hole 2 having an aperture area less than 1.05 times the maximum cross-sectional area of the particle, one particle is hardly arranged. Alternatively, in a hole 2 having an aperture area exceeding 2.63 times the maximum cross-sectional area of the particle, a plurality of particles is often arranged. One base 1 may be provided with a plurality of holes 2 differing in aperture area.

The base 1 is not particularly required to have a uniform thickness. However, its thickness in the vicinity of the hole 2 can be 0.2 times to 1.9 times the maximum size of each particle to be held for the purpose of arranging only one particle 3 in each hole 2. The maximum size of the particle 3 refers to the diameter of the maximum cross-sectional area of, for example, a sphere or almost sphere. At a thickness less than 0.2 times the maximum size (or diameter for a sphere) of the particle 3, the particle cannot be held stably because the liquid is held therein in a small amount relative to the particle. As a result, an increasing rate of particles drops off when arranged. Alternatively, at a thickness exceeding 1.9 times the maximum size (or diameter for a sphere) of the particle 3, a plurality of particles 3 is held in one hole 2 with increasing probability.

The sheet has a plurality of through-holes 2 in the base 1. The through-holes 2 may be disposed in any region of the base 1 and can be arranged regularly in terms of test or automated handling for cells described later. Moreover, the number and density of the holes 2 arranged in one base 1 are not limited by any means. From an operational standpoint, holes 2 of approximately 1 mm in diameter can be arranged at a density ranging from 10 holes/cm² to 100 holes/cm². Specifically, at a density less than 10 holes/cm², the base has a wide area other than the holes, and a water droplet tends to remain in isolation in this region. Thus, the particle is arranged, together with the water droplet, in the region other than the holes 2, resulting in reduced arrangement efficiency. Alternatively, at a density exceeding 100 holes/cm², the contamination problem in such a way that the liquid 4 holding the particle 3 is mixed with that held in its adjacent hole 2 tends to occur in subsequent handling, due to insufficient distance between the holes 2. Specifically, the ratio between the area of the sheet and the areas of the holes 2 (aperture ratio) can fall within a range of 7.9% to 78.5%.

In the present invention, not all of the holes 2 are necessarily required to hold the particle 3. However, as the rate of holes 2 holding the particle 3 is extremely reduced, the efficiency of subsequent test for cells is reduced.

(Method for Holding Particles)

The use of the sheet provided with a plurality of through-holes 2 enables the cell-supported particles 3 to be conveniently arranged. Specifically, the cell-supported particles 3 can be arranged easily one by one in the holes 2 together with a liquid by bringing a liquid containing the cell-supported particles 3 dispersed therein (suspension liquid) into contact with the sheet. The major feature of the present invention is that one particle 3 is held, together with the liquid 4, in each through-hole 2. Thus, the present invention is distinctly differentiated from conventional techniques known in the art in which a protrusion is formed in each through-hole 2 such that the partial cross-section of the hole is smaller in size than each particle or particles are supported depending on a shape by combining holes with bottom plates.

The liquid 4 containing the particles 3 (hereinafter, also referred to as a “particle-containing liquid”) is brought into contact with the sheet by a method that can be selected from a method which includes pouring the particle-containing liquid 4 to the sheet from above and a method which includes dipping the sheet into the particle-containing liquid 4. A slit-type coater known in the art may be used as one method which includes pouring the particle-containing liquid 4 thereto from above. In such a case, the particle-containing liquid 4 can be placed uniformly all over the surface of the sheet from the slit die. Then, an excess of the liquid 4 after the contact can be eliminated easily by tilting the sheet, wiping the liquid off the back thereof using a blade, or blowing air thereon. However, when air is blown thereon, the air pressure or angle of blowing must be controlled so as not to blow off the particles 3 held in the holes 2. A redundant liquid 4, if any, connecting liquids 4 in a plurality of holes 2 tends to cause contamination between the holes 2. This also tends to cause the particles 3 to be held in a site other than the holes 2. Therefore, as few droplets as possible can be allowed to remain in the site other than the holes 2. Examples of the liquid 4 include a cell culture solution containing a large number of particles 3 suspended therein.

At least one surface of the sheet can be a hydrophobic surface. Owing to the hydrophobic surface, an excess of the liquid 4 can be removed easily because the region other than the holes 2 is repellent to the aqueous liquid 4. In this case, the hydrophobic surface may have a contact angle of approximately 90 degree or larger relative to the liquid 4.

For holding each particle 3 by the surface tension of the liquid 4 in the sheet, the liquid can have a surface tension of approximately 25 mN/m or more. A liquid 4 having a surface tension less than 25 mN/m may hardly permit the particle 3 to be held in each hole 2 together therewith and often cause the particle 3 to drop off even by slight oscillation. The liquid 4 held together with the particle 3 in the sheet can be an aqueous liquid 4 that has high affinity for the cell-supported particle 3 and easily adheres to the inner wall of the hole 2.

Examples of the aqueous liquid 4 include water and water-soluble liquids (e.g., alcohol, glycol solvents and glycerin), and aqueous solutions containing these water-soluble liquids. When the aqueous solutions are used, these aqueous solutions can particularly contain 50% or more water. Furthermore, at least one agent can be selected from humectants, surface tension adjusters and thickeners and added thereto for the purpose of preventing vaporization of the particle 3-holding liquid 4 from the sheet or for the purpose of stabilizing the particle 3 holding.

Examples of the humectants include: polyhydric alcohols such as glycerin, propylene glycol, butylene glycol and sorbitol; mucopolysaccharides such as hyaluronic acid and chondroitin sulfate; and protein hydrolysates such as soluble collagen, elastin and keratin. These humectants can be used alone or as a mixture of some of them.

Examples of the surface tension adjusters include water-soluble anionic, cationic, amphoteric or nonionic surfactants. One or more of these surface tension adjusters can be added. However, the surface tension adjuster(s) are added in an amount that permits the liquid to have a surface tension of 25 mN/m or more.

Examples of the thickeners include water-soluble polymer compounds including: starches such as acid-modified starch, enzyme-modified starch, thermo-chemically modified starch, cationic starch, amphoteric starch and esterified starch; cellulose derivatives such as carboxymethylcellulose, hydroxyethylcellulose and ethylcellulose; natural or semisynthetic polymers such as casein, gelatin and soybean proteins; and polyvinyl alcohols such as completely or partially saponified polyvinyl alcohol, acetoacetylated polyvinyl alcohol, carboxy-modified polyvinyl alcohol, olefin-modified polyvinyl alcohol and silyl-modified polyvinyl alcohol. At least one water-soluble polymer compound can be selected appropriately from among these thickeners and used.

In the present invention, the liquid used can have a viscosity of 0.1 Pa·s (pascal second) or more. If necessary, the thickener(s) may be added thereto. The liquid may be controlled to have a salt concentration or pH suitable for the cells. In such a case, salts such as sodium chloride or various pH adjusters, or antiseptics or antimicrobial agents may be added appropriately thereto.

### Second Embodiment

A cell testing method of the present invention includes: holding cells by the cell holding method; and administering a substance to the cells to conduct a test on the cells. The cell testing method utilizes the sheet provided with a plurality of through-holes 2 and therefore has the following advantages, compared with conventional wells having the bottom: i) the particles 3 can be arranged easily; ii) the particles 3 can be taken out easily; iii) the particles 3 have no contact with bottom plates and are therefore unaffected by stimuli or the like from the bottom plates in cell test; iv) the liquid 4 in each hole 2 has a large area coming in contact with the outside air to provide easy gas exchange; and v) observation procedures in the absence of bottom plates are free from noise factors such as reflected light or autofluorescence caused by the bottom plates.

(Administration of Substance)

The procedure of administering a substance to the cells on the particles held in the holes 2 together with the liquid 4 in the sheet to conduct a test on the cells will be described in detail.

In this embodiment, the substance administered to the cells encompasses, but not limited to, organic or inorganic chemical substances, metals and compounds thereof, substances constituting living bodies, biologically active substances derived from living bodies, DNA, bacteria, viruses, and complexes with those chemical substances, and mixtures of a plurality of chemical substances. The administration of the substance can be performed by applying a droplet containing one or more of these substances to the holes 2 in the sheet.

In this embodiment, the substance administered to the cells can be examined for the presence or absence of its effect. When the effect is present, changes in the effect caused by varying doses or time-dependent changes in the effect can be examined. The substance can be administered by a method which includes dissolving or dispersing the substance in a solvent such as water or an organic solvent and administering this liquid. Such a liquid can be discharged in a droplet form and administered directly onto the cells held in the holes 2 in the sheet. Examples of an apparatus for preparing the liquid in a droplet form include micropipettes, microdispensers, and apparatuses which discharge droplets from a nozzle using an energy-generating device, i.e., discharge apparatuses using an inkjet method. The discharge apparatuses using an inkjet method can be used suitably in terms of microdroplets that can be discharged therefrom. Among inkjet methods, particularly, a thermal inkjet or piezoelectric inkjet method can be used.

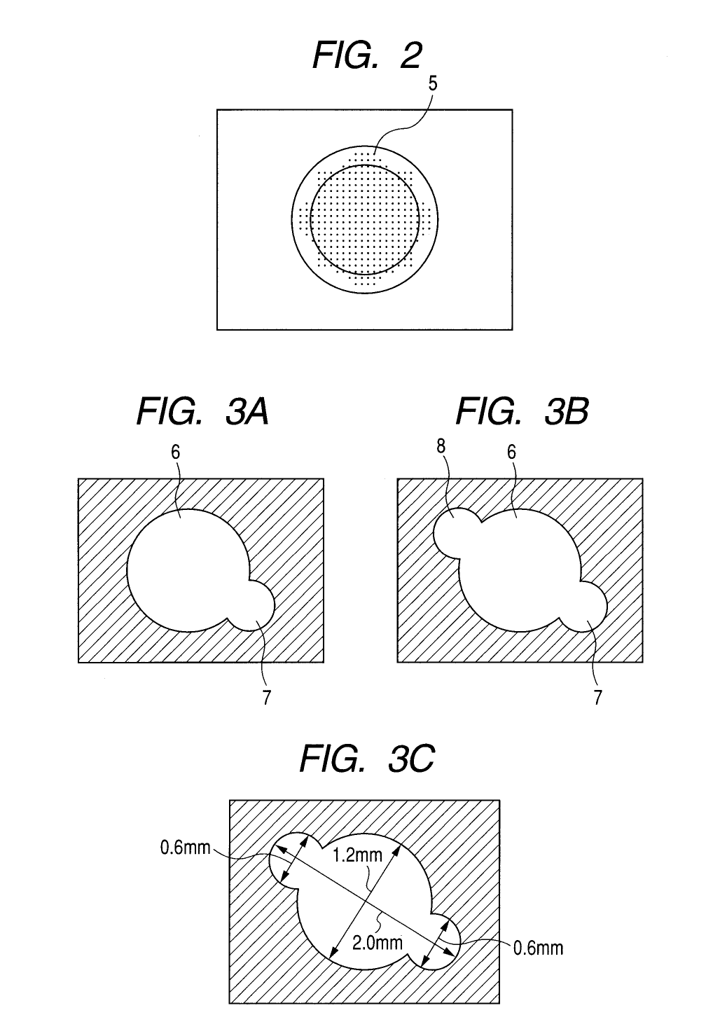

In this embodiment, the liquid can be administered in a droplet form having a volume of 100 pl (picoliter) or smaller per drop. Specifically, a droplet administered in a volume larger than 100 pl to the particle 3 held in each hole 2 together with the liquid 4 in the sheet may cause the particle 3 to drop off due to the discharge pressure of the droplet. Moreover, microdroplets can be added in a dispersed state to the whole surface of the area of each hole 2 to avoid localized administration to the particles 3. In this case, 100 or more drops can be administered in a dispersed state to one hole 2. This enables the substance to reproducibly act on the cells in the area. FIG. 2 schematically illustrates the positions of droplets in exemplary administration. FIG. 2 is a schematic plane view of the sheet, wherein positions 5 at which the droplets are spotted are illustrated.

According to the cell testing method of the present invention, the correlation of the administered substance with the cells can be tested. Specifically, this test involves, for example, (1) evaluation of morphological change of the cells, (2) quantification of substances incorporated into the cells, (3) quantification of substances synthesized in the cells, (4) evaluation of morphology or quantification of substances based on the stained cells, (5) detection of signals based on reporter genes, (6) quantification of substances by radiation dose measurement, (7) quantification of substances by fluorescence intensity measurement, (8) quantification of substances by luminescence intensity measurement, and (9) quantification of substances by absorbance measurement.

(Liquid Exchange)

In this embodiment, the liquid in the holes 2 holding the cell-supported particles 3 can be exchanged for the purpose of controlling the duration of the action of the substance administered to the cells or for the purpose of removing substances secreted from the cells. In this case, the liquid exchange must be performed smoothly without causing the particles 3 held in the holes to drop off the holes 2. For smoothly performing the liquid exchange, the sheet can be provided with a liquid exchange site communicating with each hole 2 holding the particle 3.

FIGS. 3A, 3B and 3C illustrate one example of the sheet provided with a liquid exchange site. In FIGS. 3A, 3B and 3C, the sheet is provided with a liquid exchange site 7 communicating with each through-hole 6 capable of holding one particle 3. The sheet may further be provided with a plurality of liquid exchange sites 7 and 8 communicating with each hole to more efficiently perform liquid exchange. The liquid exchange site 7 can be a hole having an area smaller than that of the hole 6 holding the particle 3. Specifically, a liquid exchange site 7 having an area equal to or larger than that of the hole 6 holding the particle 3 hardly achieves the liquid exchange of interest because the particle is held in this liquid exchange site. Moreover, the liquid exchange site can be formed by not a through-hole but a depression (well) having the bottom.

In any case, free passage of a liquid must be established between the through-hole 2 capable of holding the particle 3 and the liquid exchange site. As an example, a method for more smoothly performing liquid exchange specifically involves adding dropwise a liquid intended for exchange to the liquid exchange site 7, while aspirating a liquid from the liquid exchange site 8. As a result, the liquid exchange is smoothly performed without causing large variations in liquid volume. The liquid exchange site 8 may be connected to a tube or thin wire so as to spontaneously discharge a redundant liquid downward.

In this embodiment, the cells supported by the particles 3 can be cultured in the holes 2 in the sheet. Accordingly, this embodiment can encompass a culture method using a plurality of cells, the method including holding cell-supported particles 3 into a sheet 9 having through-holes and then culturing the cells in the holes 2 in the sheet 9 having through-holes. This cell culture procedure can further include administering a substance (e.g., a nutritional substance for sustaining cell survive) to each cell for cell growth.

For the cell culture, it is important to control an environment surrounding the sheet having the holes 2 holding the cells to a predetermined environment. Specifically, the temperature can be adjusted to that suitable for each cell growth. For cell growth, it is also important to minimize vaporization of the liquid 4 in the holes 2 in the sheet. Thus, the relative humidity surrounding the holes is kept at 60% or more, particularly, 80% or more. A life-sustaining material such as water may be added by the administration method into the holes in the sheet for the purpose of sustaining cell growth. During the course of the growth process, a chemical substance, a cell growth-sustaining material, and the like can be administered additionally by a method such as steam, spray, mist, micropipetting, or inkjet discharge apparatuses.

### Third Embodiment

The third embodiment of the present invention provides a cell handling apparatus including at least a holding unit for holding a sheet provided with a plurality of through-holes and further including at least one of the following units (A) to (G): (A) an arranging unit for arranging cell-supported particles to the holes in the sheet; (B) a controlling unit for controlling an environment surrounding the sheet to a predetermined environment; (C) an applying unit for applying a droplet to the holes in the sheet; (D) an observing unit for observing the states of the particles held in the holes in the sheet; (E) a transporting unit for transporting, from the sheet, the particles held in the holes in the sheet; (F) a recognizing unit for recognizing the positions of the holes in the sheet; and (G) a liquid exchange unit for performing liquid exchange in the holes in the sheet.

FIG. 7 schematically illustrates the cell handling apparatus of this embodiment. The arranging unit of this embodiment includes a movable holder 10. A through-hole array sheet 9 holding cell-supported particles 3 is fixed to the movable holder 10. The movable holder 10 can move the through-hole array sheet 9 in the two axial directions of X and Y axes. In this context, the Y direction represents a direction perpendicular (vertical direction on paper surface) to an X direction 11 in FIG. 7.

The applying unit of this embodiment serves as a unit for allowing a chemical substance to act on the cells and includes an inkjet apparatus 14 that is movable in one axial direction (X axis) and discharges 8 pl of a droplet.

The observing unit of this embodiment includes, for example, a lighting portion 22 and a monitor 15 and serves as a unit for capturing on images the state and change of the cells.

Specifically, for cell morphology, the cells held in the sheet are observed by irradiating the particles 3 held in the through-hole array sheet 9 with a visible light (white light at a wavelength of 300 to 900 nm) from the lighting portion 22, while taking a bright-field image by a CCD camera (image pickup portion) 23 provided below the through-hole array sheet 9. The CCD camera (image pickup portion) 23 may be provided above the through-hole array sheet 9, i.e., on the same side as the lighting portion 22. Alternatively, the apparatus can further include a lens system between the CCD camera and the cells to observe the details of the cells under magnification.

This embodiment can also include a unit for observing fluorescence from the cells. A fluorescence image may be taken using the CCD camera or the like by irradiating the cells held in the sheet with an excitation light at a wavelength of 480 to 490 nm diffracted from an excitation light source. The taken image is confirmed in the monitor 15 and stored in a control portion 16. In addition to a camera or CCD, for example, a scanner may be used as the fluorescence image pickup unit. By this irradiation of the cells with an excitation light, the inside of the cells can be rendered luminescent. In this state, an image of the cells can be taken to easily detect the luminescence site. Moreover, the cells can be observed in more detail by superimposing, in an image processing unit, the bright-field image obtained by visible light irradiation and the fluorescence image obtained by excitation light irradiation.

The transporting unit of this embodiment serves as a unit for easily transporting the particle 3 from within the particular hole 2 in the through-hole array sheet 9 to another container. Specific examples of the transporting unit that can be used include a method which includes directly pressing the particle 3 using a rod-like pin and a method which includes blowing air thereon using air pressure. Alternatively, hydraulic pressure may be used instead of air. Particularly, the method using air pressure can be used for causing little damage on the cells supported by the particles 3. A container may further be disposed below the through-hole array sheet 9. In such a case, the particular particle can be transported conveniently at a high speed to the particular container (e.g., a recovery container 21).

The recognizing unit of this embodiment includes a sensor portion 17. Specifically, the control portion 16 receives positional information from the sensor portion 17 (recognizing unit (F)) that recognizes the positions of the holes 2 in the through-hole array sheet 9 and controls the positions of the holes in the through-hole array sheet via the movable holder 10.

The liquid exchange unit of this embodiment includes a liquid exchange apparatus 29 that can be moved in the direction of a Z axis 28. The liquid exchange apparatus 29 is equipped with two glass capillaries 24 (outer diameter: 0.68 mm, inner diameter: 0.20 mm), one of which is connected via a feed pump 25 to a supply liquid container 26 and the other of which is connected via the feed pump 25 to a liquid waste container 27. For liquid exchange, the liquid exchange apparatus is moved in the direction of the Z axis such that the tips of the capillaries come in contact with the liquid exchange site in the through-hole array sheet 9. Next, the feed pump 25 is driven to supply a liquid in the supply liquid container while discharging a liquid. The type of a liquid to be exchanged can be selected by shifting a magnetic valve 30. Alternatively, the liquid exchange apparatus may be provided with a plurality of capillaries for simultaneous performing liquid exchange of a plurality of through-holes. FIG. 7 also illustrates a chamber 12, a temperature-humidity controlling apparatus 13, a compressed air supply apparatus 18, a magnetic valve 19 and a gas injection nozzle 20.

### Fourth Embodiment

The fourth embodiment of the present invention provides a kit for cell test or culture, including: a sheet provided with a plurality of through-holes; and particles for supporting cells, wherein each of the holes has a size that permits only one of the particles to be held therein together with a liquid.

The particles for supporting cells are not limited as long as the particles are capable of supporting cells. Examples of a material for the particles include organic polymer substances and inorganic substances. Examples of the organic polymer substances include acrylic acid polymers, styrene polymers, methacrylic acid polymers, alginate hydrogels, collagen-derived peptide hydrogels and agarose gels. Examples of the inorganic substances include silica, alumina, hydroxyapatite and magnetic iron oxide.

The particles may have a surface coated with a cell immobilization material. Examples of the cell immobilization material include: cell adhesion proteins such as collagen, fibronectin, vitronectin and laminin; and positively charged polymers such as polyethyleneimine, polyornithine and polylysine.

### Examples

Hereinafter, the present invention will be described specifically with reference to Examples. However, the present invention is not limited thereto.

### Example 1

(Holding and Observation)

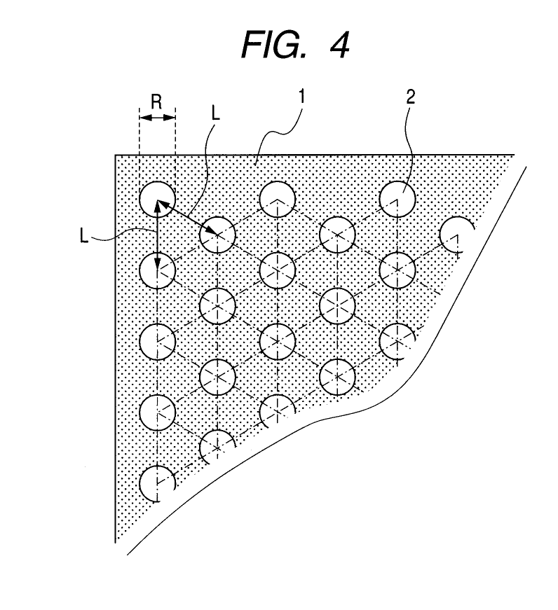

A 100 mm square stainless sheet having a thickness of 0.9 mm was processed to prepare a sheet 1 (through-hole array sheet) provided with a plurality of through-holes 2 having the following specifications. The holes 2 were round in shape as illustrated in FIG. 4 and were adjusted such that 2236 holes 2 were uniformly spaced in a close-packed manner with a hole 2 diameter R=1.2 mm and a distance L=2.25 mm between the holes 2. Cells used as targets were cells obtained by culturing human cervical cancer cell line HeLa cells (human cervix adenocarcinoma epithelial adherent ATCC CCL-2). Particles used were glass beads (SPL-1000 manufactured by UNITIKA, LTD., average particle size: 1030±30 μm, standard deviation: 25 or less, soda lime glass, absolute specific gravity: 2.5, refractive index: 1.5).

The glass beads were spread all over a Petri dish of 10 cm in diameter, and 12 mL of a DMEM medium containing 10% fetal bovine serum was added thereto. To this Petri dish, the HeLa cells were seeded and cultured under 5% carbon dioxide conditions at 37° C. to allow the HeLa cells to adhere onto the glass beads.

The live cells were fluorescently stained by the addition of an aqueous Calcein AM solution.

100 ml of a suspension liquid of the glass beads with the HeLa cells adhering on their surfaces (20 beads/ml) was gently poured to the sheet from above to cause contact therebetween. Then, by removal of a redundant liquid from the sheet, the glass bead was held in each hole in the sheet, together with the liquid present in the hole. A redundant liquid on the sheet was discarded by tilting the sheet so as not to leave a water droplet in sites other than the mouth.

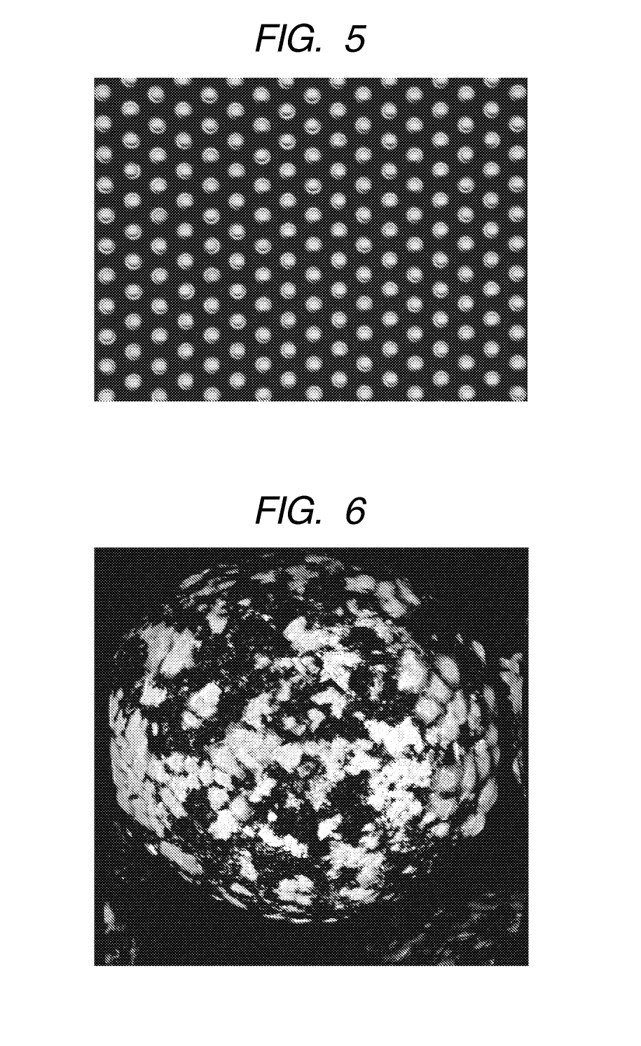

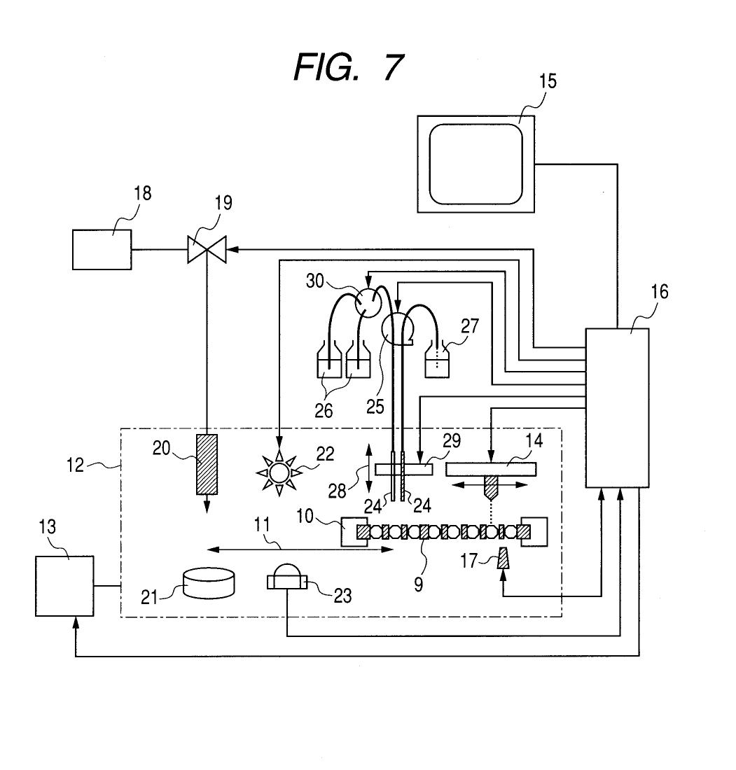

Each mouth (hole) in the thus-obtained sheet with the glass beads arranged therein was observed under a stereoscopic microscope (manufactured by Leica Microsystems, S8 APO) to observe the state of the glass bead held in each hole. As illustrated in FIG. 5, the holding of the glass beads could be observed clearly. Moreover, the HeLa cells on the glass beads were observed using a confocal microscope (Pascal Exciter manufactured by Carl Zeiss, Inc.). As illustrated in FIG. 6, the morphology of the cells on the glass beads could be observed clearly.

### Example 2

(Handling/Test, Diagnosis Apparatus)

Polystyrene series NIST Traceable Precision Particle Size Standard Particles (manufactured by MORITEX Corp., 4400A, certified average particle size: 1004±20 μm, particle density: 1.05 g/cm³) were treated with a primary amino silane coupling agent to introduce an amino group therein. After washing, the particles were dried in a drier at 45° C. An acidic solution of collagen at a concentration adjusted to 0.015% was dispensed to the particles. The particles were neutralized by the addition of a mixture alkaline solution of sodium hydroxide and sodium carbonate and left for 1 hour. The particles were washed with pure water and dried in a drier at 37° C. after removal of pure water by suction to prepare collagen-coated polystyrene particles.

The following three human breast cancer cell lines were separately cultured under 5% carbon dioxide conditions at 37° C. in a DMEM medium containing 10% fetal bovine serum: SK-BR-3 (human breast adenocarcinoma epithelial adherent, ATCC HTB-30), MDA-MB-453 (human breast adenocarcinoma epithelial adherent, ATCC HTB-131) and BT-20 (1+/0, human breast adenocarcinoma, ATCC HTB-19).

The suspension liquid of each of these three cell lines in DMEM containing 10% fetal bovine serum was collected by trypsin-EDTA treatment. These suspension liquids were separately mixed with the collagen-coated polystyrene particles in different containers and cultured with stirring (32 rpm, 37° C., 5% carbon dioxide).

A sheet (through-hole array sheet) provided with a liquid exchange site having a dimension illustrated in FIG. 3C was used. The suspension liquid containing the collagen-coated polystyrene particles was gently poured onto the sheet to cause contact therebetween. Then, by removal of a redundant liquid from the sheet, the collagen-coated polystyrene particle was held in each hole in the sheet, together with the liquid present in the hole. A redundant liquid on the sheet was discarded by tilting the sheet so as not to leave a water droplet in sites other than the mouth. This procedure was performed on these three cell lines using separate sheets.

Next, the following reagents were prepared and charged into an ink tank in an inkjet apparatus.

(1) A primary antibody solution was prepared by diluting rabbit anti-HER2/ErbB2 monoclonal antibodies (manufactured by Cell Signaling Technology, Inc.) with an antibody dilution buffer (1× TBS, 0.1% Tween-20, 5% BSA) at a ratio of 1:1000.

(2) A blocking buffer (1× TBS, 0.1% Tween-20, 5% w/v skim milk powder).

(3) A secondary antibody solution was prepared by diluting HRP-labeled anti-rabbit IgG antibodies (manufactured by Cell Signaling Technology, Inc.) with an antibody dilution buffer (1× TBS, 0.1% Tween-20, 5% BSA) at a ratio of 1:1000.

(4) A chemiluminescent substrate for HRP (ECL System manufactured by GE Healthcare).

(5) An aqueous Calcein AM solution (1 μg/mL aqueous solution).

Moreover, a washing buffer (1× TBS, 0.1% Tween-20) and a culture medium (DMEM medium containing 10% fetal bovine serum) were charged into a supply liquid container 26.

(Measurement of Parameter for the Number of Live Cells)

The aqueous Calcein AM solution was added dropwise to each cell line series. The culture medium was irradiated with a light at a wavelength of 494 nm after liquid exchange. The fluorescence intensity at 517 nm was detected using a CCD camera.

(Measurement of Parameter for HER2 Expression Level)

The procedures were sequentially performed, which involved replacement by the blocking buffer, washing, dropwise addition of the primary antibody solution, washing, dropwise addition of the secondary antibody solution, washing and dropwise addition of the chemiluminescent substrate for HRP. The luminescence intensity was detected using the CCD camera. The values of the HER2 expression level parameter normalized with the parameter for the number of live cells were BT-20<MDA-MB-453<SK-BR-3. Thus, the test evaluating and quantifying the proteins expressed in the cells (i.e., HER2 expression level) was successfully conducted. To the particles held only by the culture medium without dropwise addition of the reagents, air was injected using a gas injection nozzle 20 (transporting unit (E)) via a magnetic valve 19 from a compressed air supply apparatus 18 to drop the particles to individual recovery containers 21 disposed therebelow. The containers contained the culture medium. The recovered cells could be grown further.

### Example 3

(Variations in the Number of Cells Held in each Hole 2)

In the same way as in Example 2, three human breast cancer cell lines (BT-20, MDA-MB-453 and SK-BR-3) were separately supported by the collagen-coated polystyrene particles and cultured. To a sheet (through-hole array sheet) with 2236 holes 2 formed in a close-packed manner illustrated in FIG. 4, 100 ml of a suspension liquid of the collagen-coated polystyrene particles (20 particles/ml) was gently poured from above to cause contact therebetween. Then, by removal of a redundant liquid therefrom, the cell-supported beads were held in each hole in the through-hole array sheet.

An aqueous Calcein AM solution was added to the cells using the handling apparatus illustrated in Example 2. A parameter for the number of live cells (fluorescence intensity at 517 nm) was measured for each through-hole. Variations σ₁ in the number of cells held in each through-hole were calculated as follows.

Specifically, when the value of fluorescence intensity measured for each through-hole is defined as fᵢ, the variations σ₁ were calculated according to the following expression 2 using average fluorescence intensity calculated according to the following expression 1 (wherein n is the number of the through-holes used in the measurement. i.e., n=2236):

$$\begin{matrix}\overline{f} = \frac{1}{n} {\sum }_{i = 1}^{n} {f}_{i} & [ Expression 1 ] \\ {σ}_{1} = \sqrt{\frac{1}{n - 1} {\sum }_{i = 1}^{n} {( {f}_{i} - \overline{f} )}^{2}} & [ Expression 2 ]\end{matrix}$$

### Comparative Example 1

On the other hand, a system was prepared as Comparative Example in which cells were held in a multi-well plate by the following procedures. A suspension liquid of each cell line in DMEM containing 10% fetal bovine serum was collected by the trypsin-EDTA treatment and dispensed at a concentration of approximately 2500 cells/well to a 1536-well plate (manufactured by NUNC) without immobilizing the cells in particles. The cell suspension liquid was dispensed at a liquid volume of 10 μL containing 250 cells/μL per well. After culture under 5% carbon dioxide conditions at 37° C., a parameter for the number of live cells (fluorescence intensity at 517 nm) was measured for each well. Variations σ₂ in the number of cells held in each well were calculated as follows. Specifically, when the value of fluorescence intensity measured for each well is defined as fᵢ′, the variations σ₂ were calculated according to the following expression 4 using average fluorescence intensity calculated according to the following expression 3 (wherein n=1536):

$$\begin{matrix}{\overline{f}}^{′} = \frac{1}{n} {\sum }_{i = 1}^{n} {f}_{i}^{′} & [ Expression 3 ] \\ {σ}_{2} = \sqrt{\frac{1}{n - 1} {\sum }_{i = 1}^{n} {( {f}_{i}^{′} - {\overline{f}}^{′} )}^{2}} & [ Expression 4 ]\end{matrix}$$

The comparison between σ₁ and σ₂ measured/calculated in the different cell holding systems demonstrated σ₂>σ₁ for all the cell lines BT-20, MDA-MB-453 and SK-BR-3. This is presumably because the variations in the number of seeded cells are fixed depending on each well in the cell holding system in the multi-well plate, whereas the variations in the number of cells supported by each particle are reduced by the stirring procedure in the culture system involving mixing with the collagen-coated polystyrene particles.

Furthermore, the system using the multi-well plate requires a time for the procedure of dispensing to each well, during which the suspended state of cells is presumably altered, resulting in larger variations. Specifically, the cell holding method of the present invention enables cells to be held in 2200 or more addressable through-holes with rapidity and few variations.

The method of the present invention can be used in, for example, pharmacological activity test on compounds as drug candidates, toxicity test on chemical substances, and test on selection of anticancer agents for primary tumor cells collected by biopsy.

The present invention is not limited to the above embodiments and various changes and modifications can be made within the spirit and scope of the present invention. Therefore to apprise the public of the scope of the present invention, the following claims are made.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2009-131625, filed May 29, 2009, which is hereby incorporated by reference herein in its entirety.

What is claimed is:

1. A cell holding method comprising:
preparing a sheet provided with a plurality of through-holes; and
bringing a suspension liquid of cell-supported particles into contact with the sheet, wherein
each of the holes has a size that permits only one of the particles to be held therein together with a liquid.

2. The cell holding method according to claim 1, wherein each of the holes has a size that enables one particle to be held and does not permit two or more particles to be held.

3. The cell holding method according to claim 1, wherein each of the holes has an aperture area that falls within a range of 1.05 times to 2.63 times the maximum cross-sectional area of each of the particles to be held therein.

4. A cell testing method comprising:
holding cells by a cell holding method according to claim 1; and
administering a substance to the cells to conduct a test on the cells.

5. A cell handling apparatus comprising a holding unit for holding a sheet provided with a plurality of through-holes and further comprising at least one of the following units (A) to (G):
(A) an arranging unit for arranging cell-supported particles to the holes in the sheet;
(B) a controlling unit for controlling an environment surrounding the sheet to a predetermined environment;
(C) an applying unit for applying a droplet to the holes in the sheet;
(D) an observing unit for observing the states of the particles held in the holes in the sheet;
(E) a transporting unit for transporting, from the sheet, the particles held in the holes in the sheet;
(F) a recognizing unit for recognizing the positions of the holes in the sheet; and
(G) a liquid exchange unit for performing liquid exchange in the holes in the sheet.

6. A kit for cell test or culture, comprising:
a sheet provided with a plurality of through-holes; and
particles for supporting cells, wherein
each of the holes has a size that permits only one of the particles to be held therein together with a liquid.

7. The kit according to claim 6, wherein the particles have a surface coated with a cell immobilization material for immobilizing the cells.
